(19) United States
(12) Patent Application Publication
(10) Pub. No.: US 2025/0204820 A1
(43) Pub. Date: Jun. 26, 2025

(54) CONTACTLESS SENSOR-DRIVEN DEVICE, SYSTEM AND METHOD ENABLING ASSESSMENT OF OXYGEN SATURATION

(71) Applicant: Norbert Health, Inc., Brooklyn, NY (US)

(72) Inventors: Alexandre G. Winter, Brooklyn, NY (US); Jonathan Platkiewicz, Boulogne-Billancourt (FR); Alexis Bienvenu, Brouzet-les-Ales (FR); Xavier Ding, Bagneux (FR)

(21) Appl. No.: 18/978,634

(22) Filed: Dec. 12, 2024

Related U.S. Application Data
(60) Provisional application No. 63/613,883, filed on Dec. 22, 2023.

(51) Int. Cl. A61B 5/1455; A61B 5/00

(52) U.S. Cl. CPC A61B 5/14552; A61B 5/7203; A61B 5/7267; A61B 5/742; A61B 2576/00

(57) ABSTRACT

Embodiments of the present disclosure relate to a method of estimating peripheral oxygen saturation (SpO2) in a human subject, and devices and apparatuses configured to perform the same.

### CROSS-REFERENCE TO RELATED APPLICATION(S)

This application claims the benefit of priority of U.S. Provisional Patent Application No. 63/613,883, filed Dec. 22, 2023, the disclosure of which is hereby incorporated by reference herein in its entirety.

### BACKGROUND

Peripheral oxygen saturation (SpO2) is a well-known indicator in the assessment of respiratory health. Although SpO2 is often measured during routine examination of patients, regular and periodic measurement of SpO2 correlating a measurement of blood oxygenation is desirable for better assessing the likelihood of an adverse respiratory event.

SpO2 is defined as the fraction of oxygen-saturated hemoglobin relative to total hemoglobin (unsaturated and saturated) in the blood. In healthy adults, typical values lie above 95% (and below 100%). Any level below this range is generally considered to be an indication that the subject is in need of medical attention, and the lower the value, the more concerning the subject's condition is.

SpO2 is estimated by measuring the amount of light that is transmitted or reflected by skin tissue in two well-chosen wavelengths. In classic pulse oximetry, the incoming light is entirely controlled and highly constrained, while in remote camera-based pulse oximetry, ambient light is the input and cannot be controlled by the device.

On a homogenous skin surface, color changes induced by blood flow variations can be captured by a conventional digital camera. Blood oxygenation levels have a direct impact on the color of the skin. The different hemoglobin types (e.g., oxyhemoglobin and deoxyhemoglobin) have different color responses to light based on oxygen saturation.

Typical remote photoplethysmography (rPPG) applications rely on available commercial cameras, such as those embedded in smartphones, and do not modify its image signal processor (ISP). This approach has the advantage of being readily applicable, to a smartphone user for example, by simply downloading the appropriate software application. However, this approach comes with great technical challenges given the amount of processing incorporated in current digital cameras, especially with respect to the robustness to light variability.

Accordingly, there is a need for improved approaches, for example, to remotely assess oxygen saturation using a camera whose ISP is fully controllable.

### SUMMARY

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect, a method of estimating peripheral oxygen saturation (SpO2) in a human subject comprises: capturing, by a camera, video comprising a plurality of image frames of the face and/or a hand palm of the human subject in the presence of an ambient light source; identifying, within the plurality of image frames, a plurality of skin pixels corresponding pixels that spatially correspond to skin of the face and/or the hand palm within each of the plurality of image frames; computing, for each of the plurality of skin pixels, a time-dependent signal corresponding to blood volume change for each color channel of the skin pixel; generating, based on the time-dependent signal, a plurality of SpO2 estimates for each of the plurality of skin pixels at each frame of the captured video; and computing an overall SpO2 estimate from the plurality of SpO2 estimates.

In at least one embodiment, generating the plurality of SpO2 estimates comprises, for each of the plurality of skin pixels, applying as inputs to a machine learning model a plurality of parameters derived from the corresponding time-dependent signal.

In at least one embodiment, the machine learning model comprises a multilayer perceptron model comprising at least three hidden layers.

In at least one embodiment, prior to capturing the video, one or more performance settings of the camera are disabled, wherein the one or more performance settings are selected from bad pixel correction, Bayer domain hardware noise reduction, high-order lens-shading compensation, auto-white-balance, de-mosaic filtering, 3×3 color transformation, color artifact suppression, downscaling, or edge enhancement.

In at least one embodiment, identifying the plurality of skin pixels within the one or more image frames comprises detecting the face and/or the hand palm and extracting face landmarks and/or hand landmarks to determine bounds of the skin.

In at least one embodiment, computing, for each of the plurality of skin pixels, the time-dependent signal corresponding to blood volume change for each color channel of the skin pixel comprises computing the time-dependent signal for each of a red-channel, a green-channel, and a blue-channel of each skin pixel.

In at least one embodiment, computing the overall SpO2 estimate from the plurality of SpO2 estimates comprising applying a statistical inference to the plurality of SpO2 estimates computed over all of the identified skin pixels within each of the plurality of image frames of the captured video.

In at least one embodiment, prior to capturing the video, increasing a color depth of video capture to 10 or more bits per color channel.

In at least one embodiment, the method further comprises: displaying, by a display device, or causing to be displayed the overall SpO2 estimate.

In a further aspect, a device to estimate peripheral oxygen saturation (SpO2) in a human subject comprises: a housing; a camera disposed at least partially in the housing; and a processing device disposed within the housing, the processing device being communicatively coupled to camera. In at least one embodiment, the processing device is configured to perform the method according to any of the preceding embodiments.

In at least one embodiment, the device further comprises: a display device configured to display the overall SpO2 estimate computed according to the method of any of the preceding embodiments.

In a further aspect, a non-transitory computer-readable medium comprises instructions stored thereon, which, when executed by a processing device that is operatively coupled to a camera, causes the processing device to perform operations the method of any of the preceding embodiments.

### BRIEF DESCRIPTION OF DRAWINGS

The examples described herein will be understood more fully from the detailed description given below and from the accompanying drawings, which, however, should not be taken to limit the application to the specific examples, but are for explanation and understanding only.

FIG. 1 is a block diagram illustrating an exemplary system architecture, in accordance with at least one embodiment.

FIG. 2 is a schematic illustrating the high-level operation of a device for estimating SpO2, in accordance with at least one embodiment.

FIG. 3 illustrates an exemplary image processing pipeline, in accordance with at least one embodiment.

FIG. 4 is a process flow illustrating a hierarchy of processing performed to estimate SpO2, in accordance with at least one embodiment.

FIG. 5 is a flow diagram illustrating an exemplary method of estimating SpO2 in a human subject, in accordance with at least one embodiment.

FIG. 6 is a block diagram illustrating a computer system, in accordance with at least one embodiment.

### DETAILED DESCRIPTION

The embodiments described herein relate generally to assessing respiratory health and, more specifically, to a non-invasive device, system, and method of assessing blood oxygenation using one or more contactless sensors.

In at least one embodiment, a monitoring device comprising a plurality of contactless sensors is provided. The sensors may be configured to detect, measure, monitor, and provide alerts where appropriate, in connection with various vital signs, movement and recognized routines of an individual. Data collected by sensors may be processed in accordance with various algorithmic processes suited for measurement of SpO2. These algorithmic processes may be executed directly on the monitoring device, remote from the monitoring device or on, in some instances, a combination of both. Additionally, the foregoing sensors may be integrated in a single monitoring device unit, optionally coupled to a centralized monitoring device or any suitable combination thereof depending on the monitoring environment.

In at least one embodiment, the monitoring device may be integrated as part of an overall health monitoring system, where the system may be comprised of the monitoring device, a plurality of mobile devices associated with various individuals (e.g., patients, health care providers, family members, etc.) configured with monitoring/notifying applications (e.g., a caregiver application), one or more servers, one or more databases, and one or more secondary systems that are communicatively coupled to enable the health monitoring system described herein. Such monitoring devices, systems, and methods are disclosed in U.S. Non-Provisional application Ser. No. 17/225,313, the disclosure of which is hereby incorporated by reference herein in its entirety.

The embodiments described herein addresses the shortcomings of existing approaches by providing an improved device, system and method for measuring light reflection to calculate SpO2 and, in turn, yield an assessment/estimation of oxygen saturation. The devices, systems, and methods of the present disclosure have several advantages over conventional solutions. For example, the devices can be used in a location (e.g., home, office, store, restaurant, etc.) that is not a medical facility to determine health conditions compared to conventional solutions of visiting a medical facility to determine health conditions. The devices can be contactless compared to conventional wearable solutions that users forget to wear, users forget to charge, or are limited in incorporation of components in the conventional wearable device. A device may be a single non-intrusive device that has ease of installation compared to intrusive conventional solutions of multiple cameras particularly positioned throughout an indoor space that often require a more difficult installation.

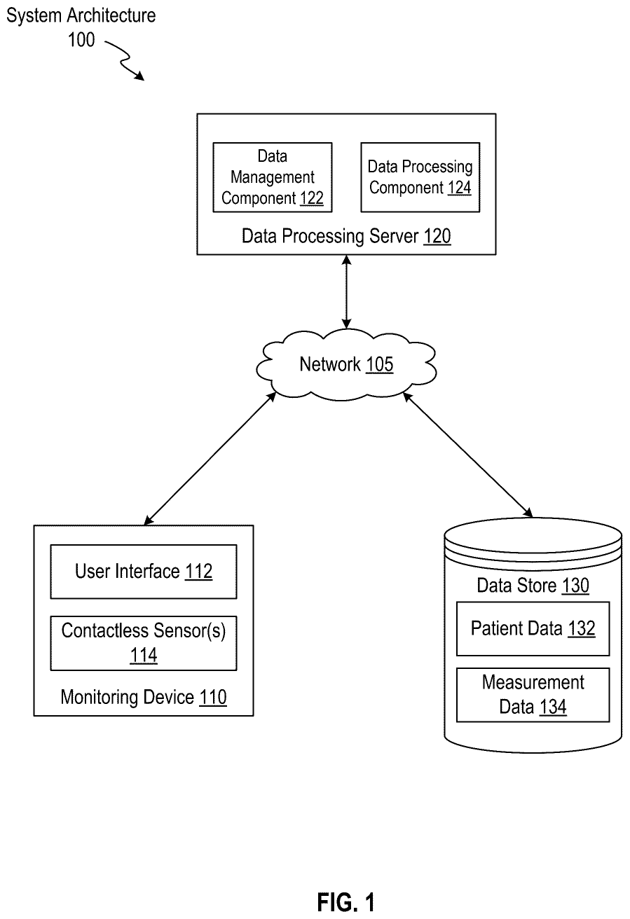

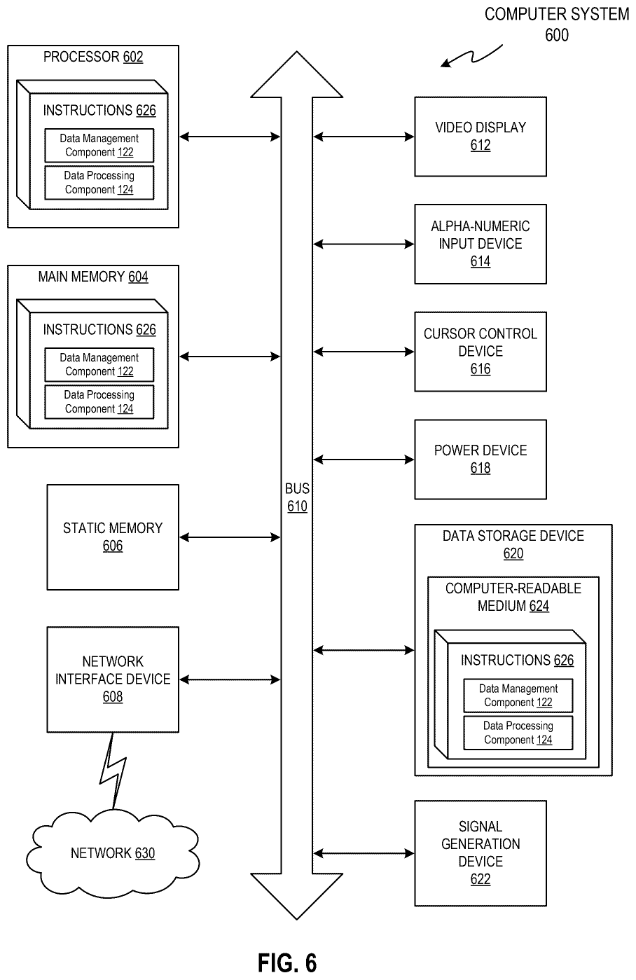

Exemplary implementations of the present disclosure are now described. FIG. 1 illustrates an exemplary system architecture 100, in accordance with at least one embodiment. The system architecture 100 includes a monitoring device 110, a data processing server 120, and a data store 130, with each device of the system architecture 100 being communicatively coupled via a network 105. One or more of the devices of the system architecture 100 may be implemented using a generalized computer system 600, described with respect to FIG. 6. The devices of the system architecture 100 are merely illustrative, and it is to be understood that other user devices, data processing servers, data stores, and networks may be present.

In one embodiment, network 105 may include a public network (e.g., the Internet), a private network (e.g., a local area network (LAN) or wide area network (WAN)), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof. Although the network 105 is depicted as a single network, the network 105 may include one or more networks operating as stand-alone networks or in cooperation with each other. The network 105 may utilize one or more protocols of one or more devices to which they are communicatively coupled.

In one embodiment, the monitoring device 110 may include a computing device such as a personal computer (PC), laptop, mobile phone, smart phone, tablet computer, netbook computer, etc. The monitoring device 110 may also be a standalone device having one or more processing devices housed therein. An individual user may be associated with (e.g., own and/or operate) the monitoring device 110. As used herein, a “user” may be represented as a single individual. However, other embodiments of the present disclosure encompass a “user” being an entity controlled by a set of users and/or an automated source. For example, a set of individual users federated as a community in a company or government organization may be considered a “user.”

The monitoring device 110 may utilize one or more local data stores, which may be internal or external devices, and may each include one or more of a short-term memory (e.g., random access memory), a cache, a drive (e.g., a hard drive), a flash drive, a database system, or another type of component or device capable of storing data. The local data stores may also include multiple storage components (e.g., multiple drives or multiple databases) that may also span multiple computing devices (e.g., multiple server computers). In at least one embodiment, the local data stores may be used for data back-up or archival purposes.

The monitoring device 110 may implement a user interface 112, which may allow a user of the monitoring device 110 to send/receive information to/from other monitoring devices (not shown), the data processing server 120, and the data store 130. The user interface 112 may be a graphical user interface (GUI). For example, the user interface 112 may be a web browser interface that can access, retrieve, present, and/or navigate content (e.g., web pages such as Hyper Text Markup Language (HTML) pages) provided by the data processing server 120. In one embodiment, the user interface 112 may be a standalone application (e.g., a mobile “app,” etc.), that enables a user to use the monitoring device 110 to send/receive information to/from other monitoring devices (not shown), the data processing server 120, and the data store 130. In at least one embodiment, the user interface 112 provides a visual readout of measurements or estimations of physiological parameters, such as SpO2, made the monitoring device 110 or in combination with another device, and/or may provide instructions to the user operating the monitoring device 110 visually, aurally, or both.

In at least one embodiment, the monitoring device 110 comprises one or more contactless sensor(s) 114. The contactless sensor(s) 114 may include one or more high-resolution visible light cameras, infrared imagers, radar sensors, microphone arrays, depth sensors, ambient temperature sensors, ambient humidity sensors, or other sensing devices. The contactless sensor(s) 114 may be at least partially disposed within a housing of the monitoring device 110 to protect the sensor. In some embodiments, the contactless sensor(s) 114 include at least a high resolution visible light camera. This camera is mostly sensitive to the visible light wavelengths range (approximately from 400 to 1000 nm).

In one embodiment, the data processing server 120 may include one or more computing devices (such as a rackmount server, a router computer, a server computer, a personal computer, a mainframe computer, a laptop computer, a tablet computer, a desktop computer, etc.), data stores (e.g., hard disks, memories, databases), networks, software components, and/or hardware components from which digital contents may be retrieved. In at least one embodiment, the data processing server 120 may be a server utilized by the monitoring device 110, for example, to process image data and provide the monitoring device 110 with resulting measurement or estimation data. In at least one embodiment, additional data processing servers may be present. In at least one embodiment, the data processing server 120 utilizes a data management component 122 to process, store, and analyze patient data 132 and measurement data 134 (which may be stored in the data store 130 or received directly from the monitoring device 110). In at least one embodiment, the data processing server further utilizes a data processing component 124 to process data obtained from contactless sensor(s) 114 and to compute measurements or estimates of one or more physiological parameters, such as SpO2. The role of the data processing component 124 will be described in greater detail below.

In at least one embodiment, the data processing component 124 utilizes a machine learning platform that may be configured to apply one or more machine learning models, for example, for the purposes of generating estimates of SpO2. In at least one embodiment, the machine learning platform may utilize models comprising, e.g., a single level of linear or non-linear operations, such as a support vector machine (SVM), or a deep neural network (i.e., a machine learning model that comprises multiple levels of linear or non-linear operations). For example, a deep neural network may include a neural network with one or more hidden layers. Such machine learning models may be trained, for example, by adjusting weights of a neural network in accordance with a backpropagation learning algorithm. In at least one embodiment, the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naïve Bayes model, or a deep learning model. In some embodiments, the machine learning model is a supervised machine learning model or an unsupervised machine learning model.

In at least one embodiment, each machine learning model may include layers of computational units (“neurons”) to hierarchically process data, and feeds forward the results of one layer to another layer so as to extract a certain feature from the input. When an input vector is presented to the neural network, it may be propagated forward (e.g., a forward pass) through the network, layer by layer (e.g., computational units) until it reaches an output layer. The output of the network can then be compared to a desired output (e.g., a label), using a loss function. The resulting error value is then calculated for each neuron in the output layer. The error values are then propagated from the output back through the network (e.g., a backward pass), until each neuron has an associated error value that reflects its contribution to the original output.

In at least one embodiment, the machine learning model is trained based on parameters derived from time-dependent signals derived from skin pixels of a subject's face and/or palm. For example, while recording video of the patient's face and/or palm(s), excess post-exercise oxygen consumption (EPOC) analysis can be performed on the subject while the subject is wearing a mask that varies the supply of oxygen. SpO2 measurements for that subject can then be correlated with the parameters derived from the time-dependent signals, which are then used to train the machine learning model.

In one embodiment, the data store 130 may include one or more of a short-term memory (e.g., random access memory), a cache, a drive (e.g., a hard drive), a flash drive, a database system, or another type of component or device capable of storing data. The data store 130 may also include multiple storage components (e.g., multiple drives or multiple databases) that may also span multiple computing devices (e.g., multiple server computers). In at least one embodiment, the data store 130 may be cloud-based. One or more of the devices of system architecture 100 may utilize their own storage and/or the data store 130 to store public and private data, and the data store 130 may be configured to provide secure storage for private data. In at least one embodiment, the data store 130 stores patient data 132, which may include health records of individual patients, biometric data, health conditions, and other information. In at least one embodiment, the data store 130 stores measurement data 134, which may include sensor data, vital signs, predictive data, alert data, or other information. The measurement data 134 may further include current data and historical data for training one or more machine learning models. Current data may be data (e.g., current sensor data, current vital signs, etc.) used to generate predictive data and/or used to re-train the one or more machine learning models. Each instance (e.g., set) of sensor data, vital signs, user information, predictive data, etc. may correspond to a respective user and/or a corresponding period of time.

Although each of the monitoring device 110, the data processing server 120, and the data store 130 are depicted in FIG. 1 as single, disparate components, these components may be implemented together in a single device or networked in various combinations of multiple different devices that operate together. In at least one embodiment, at least some of the functionality of the data processing server 120 and/or the data store 130 may be performed by the monitoring device 110, or by other devices.

Although embodiments of the disclosure are discussed in the context of oxygen saturation measurements and estimates, such embodiments are generally applicable to other types of physiological measurements and to other areas of the body.

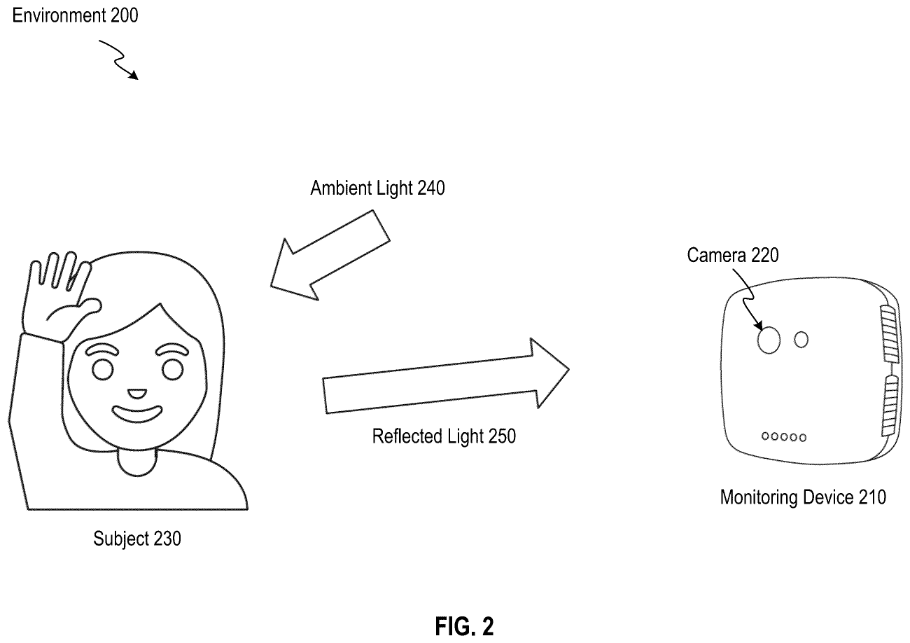

FIG. 2 is a schematic illustrating an environment 200 for which high-level operation of a device for estimating SpO2 is exemplified, in accordance with at least one embodiment. In at least one embodiment, the environment 200 includes a monitoring device 210 (which may be the same as or similar to the monitoring device of FIG. 1) that partially houses a camera 220. The camera 220 can be used to capture light 250 reflected from the face and/or hand palm of a human subject 230. In at least one embodiment, the reflected light 250 may result from ambient light 240. In at least one embodiment, the source of the ambient light 240 may be under the control of the monitoring device 210 (or another associated device) so that control can be exercised over the intensity of the light captured in the images. Color channels, corresponding to the captured red light, green light, and blue light, may be isolated from the images and used for downstream processing, as discussed below.

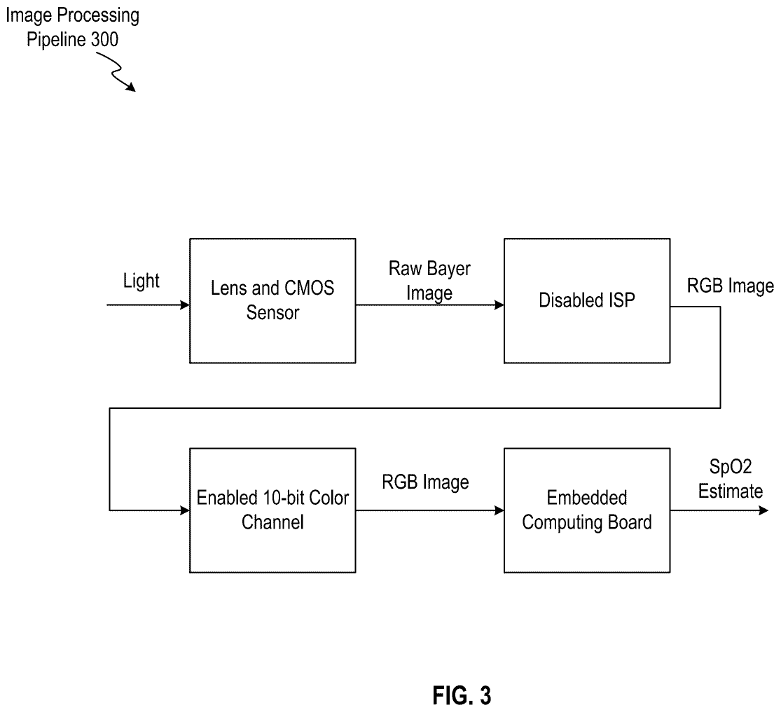

FIG. 3 illustrates an exemplary image processing pipeline 300, in accordance with at least one embodiment. Most digital cameras incorporate a complex image processing pipeline, which allows for the production of aesthetically pleasing images. In certain embodiments that utilize a camera, various adjustments may be made to the processing pipeline to cause the camera to behave more like a light measurement device rather than a photography device for the purpose of estimating SpO2. In at least one embodiment, one or more performance settings associated with the image signal processor (ISP) of the camera may be disabled, including bad pixel correction, Bayer domain hardware noise reduction, high-order lens-shading compensation, auto-white-balance, de-mosaic filtering, 3×3 color transformation, color artifact suppression, downscaling, or edge enhancement. In at least one embodiment, two or more, three or more, four or more, five or more, or all of the aforementioned performance settings are disabled. In such embodiments, the acquired pixel values of the raw Bayer image may be closer to the true photon count compared to the results produced by the default ISP performance settings. In at least one embodiment, 10 or more bits per color channel of the camera is measured, instead of the conventional 8-bit, to capture finer chromatic temporal variation. In at least one embodiment, computation of SpO2 measurements may be performed by an embedded computing board based on an RGB as input, the embedded computing board configured for implementing the methodologies described below.

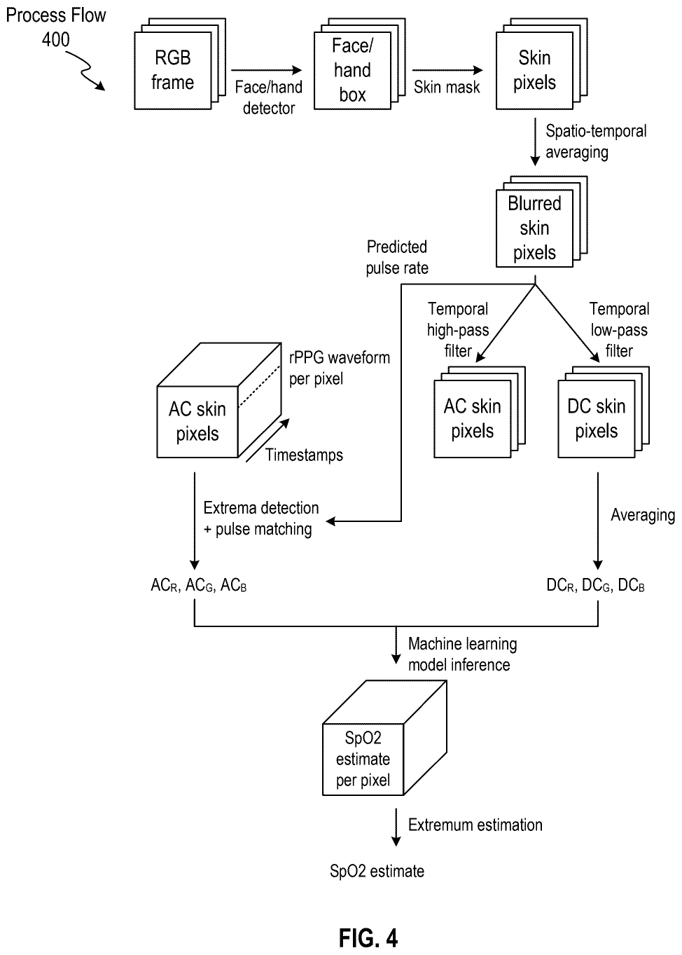

FIG. 4 is a process flow 400 illustrating a hierarchy of processing performed to estimate SpO2 according to certain embodiments. The process may be initiated in response to determining that a subject is in close physical proximity (e.g., within 1 to 2 meters) to the monitoring device (e.g., the monitoring device 210), and adopts a frontal, stable position, and their face and/or hand palm are detected and tracked by processing the frames captured by a camera (e.g., the camera 220). The face and/or hand palm of multiple subjects can be simultaneously detected in a single frame, and tracked independently over subsequent frames. In addition, the face landmarks and/or hand palm landmarks of multiple subjects can be simultaneously detected in a single frame, and tracked independently over subsequent frames.

In at least one embodiment, the pixels of the face skin and/or the hand palm skin are detected. The face bounding box and/or hand palm bounding box, as well as the face landmarks and/or hand palm landmarks, can be leveraged to constrain the area in the image for skin pixel detection. In at least one embodiment, this approach is used to generate a skin mask, which is the set of contiguous pixels identified as part of the skin (referred to as “skin pixels”). In at least one embodiment, the skin mask remains constant from frame to frame of the video under the assumption that the subject's face and/or hand palm have not deviated or have deviated minimally from their original positions and orientations. In at least one embodiment, the skin mask may be updated periodically (e.g., every one frame, every two frames, etc.) to account for drive in the positions/orientations of the subject's face and/or hand palm. In at least one embodiment, SpO2 estimates derived from skin pixels may be rejected when computing a final SpO2 estimate if it is determined that the skin pixels at any point during the measurement fell outside of one or more computed skin masks. In at least one embodiment, a movement detection algorithm may be performed to detect blurred pixels indicative of movement of the subject's face and/or palm. In at least one embodiment, the estimation process is reset in response to a determination that the subject's movement may adversely impact the estimations. In at least one embodiment, the subject's motion can be quantified by the coordinates of the bounding boxes corresponding to the detected face or hand palm, respectively, over a sequence of frames.

In at least one embodiment, the motion of the face and the motion of the hand palm are evaluated independently. For example, if the face has been determined to have moved significantly, the SpO2 estimation process on the face is reset, regardless of the hand palm evaluation. Similarly, if the hand palm has been determined to have moved significantly, the SpO2 estimation process on the hand palm is reset, regardless of the face evaluation.

In at least one embodiment, each pixel of the segmented skin area is processed, independently of the other pixels. In at least one embodiment, all of the skin pixels are processed in parallel.

In at least one embodiment, a time-dependent PPG signal corresponding to blood volume change is extracted at each skin pixel by collecting the values on their corresponding three color channels over time. Frequency filtering may be performed by applying low-pass filters and/or high-pass filters to each channel component per skin pixel to isolate the pulsatile component of the blood volume change that is causally related to the subject's heart beat rhythm. Frequency filtering may further allow for the extraction of the non-periodic component of the blood volume change that is indicative of the amount of blood present in the tissues at all times.

In at least one embodiment, following the frequency filtering, each resulting pulsatile signal is segmented into pulses, characterized by a sharp upstroke and a more gradual downstroke including a potential secondary peak. The amplitude of the pulse is referred to as “AC,” and the average value of the non-periodic signal in the pulse interval is referred to as “DC.”

In at least one embodiment, the estimated heart rate is used to detect and segment each pulse of the isolated pulsatile signal. The pulses that are synchronous on multiple color channels can be grouped into a multi-dimensional pulse. In at least one embodiment, extrema detection is performed to detect in the PPG signal where the peaks and valleys are pulse-matching, which may be used to ensure that a pulse on the R channel is substantially synchronous with a pulse on the B and/or G channels. If the pulses from each channel are substantially synchronous, then they are grouped together; otherwise, they may be rejected from being used in SpO2 estimation.

In at least one embodiment, the values of AC and DC, algebraic combination of the AC and DC components (e.g., ratios of AC to DC) on each color channel, and/or the absolute duration on each color channel of a multi-dimensional pulse can be used as input parameters to a machine learning model. In at least one embodiment, the machine learning model can output a probability that SpO2 takes one of its physiologically plausible values, considered to range from 70% to 100% in a discrete fashion. In at least one embodiment, a single machine learning model is used for all the processed pixels. In at least one embodiment, the machine learning model may be a multilayer perceptron with at most three hidden layers. In at least one embodiment, parameters used as inputs can be extracted from ten or fewer consecutive pulses of the same pixel, ten or fewer pixels of the same frame, or both. In at least one embodiment, the parameters are concatenated into a single vector. In at least one embodiment, the parameters can be implicitly extracted from the time-dependent signal using a convolutional neural network, provided enough training samples have been collected. Exemplary parameters used as input to the machine learning model can include, but are not limited to:
AC_{R}, AC_{G}, AC_{B},
DC_{R}, DC_{G}, DC_{B},
AC_{R}/DC_{R}, AC_{G}/DC_{G}, AC_{B}/DC_{B},
(AC_{B}/DC_{B})/(AC_{R}/DC_{R}),
(AC_{G}/DC_{G})/(AC_{R}/DC_{R}),
Δt_{R}, Δt_{G}, Δt_{B},
   or combinations thereof, where R, G, and B subscripts represent red, green, and blue channels, respectively, and Δt represents pulse duration for an individual channel.

In at least one embodiment, an SpO2 estimate is chosen as the value for which the machine learning model output probability is maximal. In the course of a few-second-long health check session, numerous values of SpO2 are implicitly estimated, which may correspond to as many as the number of identified skin pixels multiplied by the number of frames in the captured video. The estimated values can be aggregated and stored into a single list over a certain time duration. For a health check session, a single SpO2 estimate may be given by the most likely value in this list (e.g., the most frequent value among all stored values), or via a different statistical inference method as would be appreciated by those of ordinary skill in the art.

SpO2 data may be stored (e.g., in the patient data 132 and/or the measurement data 134) to compile a profile of historical measurements for a subject. The stored SpO2 data can be retrieved, for example, by a healthcare professional examining changes in oxygen saturation as part of an overall respiratory assessment.

It is envisioned that the foregoing monitoring may be deployed in various settings. In one embodiment, the monitoring device may be deployed in a subject's home setting, wherein the SpO2 measurements may be conducted on a preconfigured schedule. For example, the monitoring device may be configured to notify the subject when it is time to measure SpO2 as part of a daily monitoring routine for assessing heart health, prompting the subject to stand in close proximity to the monitoring device for sensor data acquisition. In another embodiment, the monitoring device may be configured for deployment in an environment where a subject is in a fairly stationary position. For example, the monitoring device may be implemented in a car dashboard to monitor driver health, placed above hospital beds, implemented in a self-service health checkup station, or any other applicable health monitoring setting.

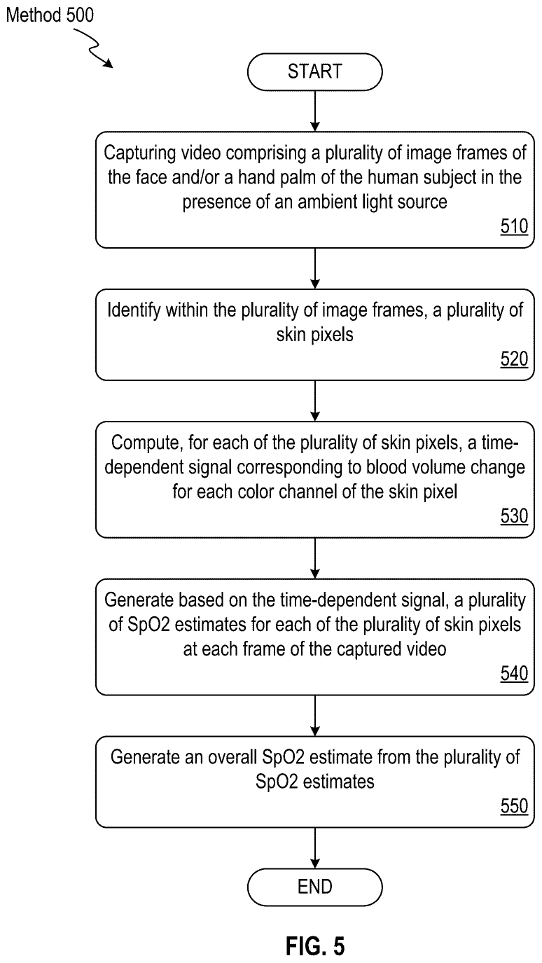

FIG. 5 is a flow diagram illustrating an exemplary method 500 of estimating SpO2 in a human subject according to certain embodiments. The method 500 can be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In at least one embodiment, the method 500 is implemented at least partially by a data processing server, such as the data processing server 120 utilizing the data management component 122 and the data processing component 124. In at least one embodiment, the method 500 may be performed, in part, by the monitoring device 110 or the data processing server 120. In at least one embodiment, a non-transitory computer-readable storage medium stores instructions that when executed by a processing device (e.g., of the data processing server 120) cause the processing device to perform method 500.

For simplicity of explanation, the method 500 is depicted and described as a series of acts. However, acts in accordance with this disclosure can occur in various orders and/or concurrently and with other acts not presented and described herein. Furthermore, not all illustrated acts may be performed to implement the method 500 in accordance with the disclosed subject matter. In addition, those skilled in the art will understand and appreciate that the method 500 could alternatively be represented as a series of interrelated states via a state diagram or events.

At block 510, a processing device (e.g., implementing the data processing component 124) causes a camera (e.g., the camera 220 of the monitoring device 110) to capture video comprising a plurality of image frames of the face and/or a hand palm of the human subject (e.g., the subject 230) in the presence of an ambient light source (e.g., a source of ambient light 240).

In at least one embodiment, prior to capturing the video, one or more performance settings of the camera are disabled (e.g., as discussed with respect to FIG. 3). In at least one embodiment, the one or more performance settings are selected from bad pixel correction, Bayer domain hardware noise reduction, high-order lens-shading compensation, auto-white-balance, de-mosaic filtering, 3×3 color transformation, color artifact suppression, downscaling, or edge enhancement. In at least one embodiment, prior to capturing the video, increasing a color depth of video capture to 10 or more bits per color channel.

At block 520, the processing device identifies a plurality of skin pixels within the plurality of image frames. For example, the plurality of skin pixels may be identified by generating a skin mask for each of the face and/or hand palm identified within the video. In at least one embodiment, identifying the plurality of skin pixels within the one or more image frames comprises detecting the face and/or the hand palm and extracting face landmarks and/or hand landmarks to determine bounds of the skin.

At block 530, the processing device computes, for each of the plurality of skin pixels, a time-dependent signal corresponding to blood volume change for each color channel (e.g., the R-channel, G-channel, and B-channels) of the skin pixel.

At block 540, the processing device generates, based on the time-dependent signal, a plurality of SpO2 estimates for each of the plurality of skin pixels at each frame of the captured video. In at least one embodiment, one or more of the SpO2 estimates may be rejected, for example, if it is determined that there were interruptions in a given time-dependent signal, if not enough synchronous pulses of other skin pixels in the same image frame were detected, or both.

At block 550, the processing device generates an overall SpO2 estimate from the plurality of SpO2 estimates. In at least one embodiment, generating the plurality of SpO2 estimates comprises, for each of the plurality of skin pixels, applying as inputs to a machine learning model a plurality of parameters derived from the corresponding time-dependent signal. In at least one embodiment, the machine learning model comprises a multilayer perceptron model comprising at least three hidden layers. In at least one embodiment, computing the overall SpO2 estimate from the plurality of SpO2 estimates comprising applying a statistical inference to the plurality of SpO2 estimates computed over all of the detected skin pixels within each of the plurality of image frames of the captured video.

In at least one embodiment, the method 500 further comprises displaying, by a display device, or causing to be displayed the overall SpO2 estimate.

FIG. 6 illustrates a diagrammatic representation of a machine in the exemplary form of a computer system 600 within which a set of instructions (e.g., for causing the machine to perform any one or more of the methodologies discussed herein) may be executed. In alternative embodiments, the machine may be connected (e.g., networked) to other machines in a LAN, an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Some or all of the components of the computer system 600 may be utilized by or illustrative of at least some of the devices of the system architecture 100, such as the monitoring device 110, the data processing server 120, and the data store 130.

The exemplary computer system 600 includes a processing device (processor) 602, a main memory 604 (e.g., read-only memory (ROM), flash memory, dynamic random access memory (DRAM) such as synchronous DRAM (SDRAM) or Rambus DRAM (RDRAM), etc.), a static memory 606 (e.g., flash memory, static random access memory (SRAM), etc.), and a data storage device 620, which communicate with each other via a bus 610.

Processor 602 represents one or more general-purpose processing devices such as a microprocessor, central processing unit, or the like. More particularly, the processor 602 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 602 may also be one or more special-purpose processing devices such as an ASIC, a field programmable gate array (FPGA), a digital signal processor (DSP), network processor, or the like. The processor 602 is configured to execute instructions 626 for performing the operations and steps discussed herein, such as operations associated with the data management component 122 and/or the data processing component 124.

The computer system 600 may further include a network interface device 608. The computer system 600 also may include a video display unit 612 (e.g., a liquid crystal display (LCD), a cathode ray tube (CRT), or a touch screen), an alphanumeric input device 614 (e.g., a keyboard), a cursor control device 616 (e.g., a mouse), and/or a signal generation device 622 (e.g., a speaker).

Power device 618 may monitor a power level of a battery used to power the computer system 600 or one or more of its components. The power device 618 may provide one or more interfaces to provide an indication of a power level, a time window remaining prior to shutdown of computer system 600 or one or more of its components, a power consumption rate, an indicator of whether computer system is utilizing an external power source or battery power, and other power related information. In at least one embodiment, indications related to the power device 618 may be accessible remotely (e.g., accessible to a remote back-up management module via a network connection). In at least one embodiment, a battery utilized by the power device 618 may be an uninterruptible power supply (UPS) local to or remote from computer system 600. In such embodiments, the power device 618 may provide information about the power level of the UPS.

The data storage device 620 may include a computer-readable storage medium 624 on which is stored one or more sets of instructions 626 (e.g., software) embodying any one or more of the methodologies or functions described herein. The instructions 626 may also reside, completely or at least partially, within the main memory 604 and/or within the processor 602 during execution thereof by the computer system 600, the main memory 604 and the processor 602 also constituting computer-readable storage media. The instructions 626 may further be transmitted or received over a network 630 (e.g., the network 105) via the network interface device 608.

In one embodiment, the instructions 626 include instructions for implementing the functionality of the data processing server 120, as described throughout this disclosure. While the computer-readable storage medium 624 is shown in an exemplary embodiment to be a single medium, the terms “computer-readable storage medium” or “machine-readable storage medium” should be taken to include a single medium or multiple media (e.g., a centralized or distributed database, and/or associated caches and servers) that store the one or more sets of instructions. The terms “computer-readable storage medium” or “machine-readable storage medium” shall also be taken to include any transitory or non-transitory medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “capturing,” “receiving,” “determining,” “detecting,” “providing,” “combining,” “training,” “obtaining,” “identifying,” “computing,” “estimating,” “causing,” “generating,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

In the above description, numerous details are set forth. It will be apparent, however, to one of ordinary skill in the art having the benefit of this disclosure, that embodiments may be practiced without these specific details. In some instances, well-known structures and devices are shown in block diagram form, rather than in detail, in order to avoid obscuring the description.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

Embodiments also relate to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, such as, but not limited to, any type of disk including floppy disks, optical disks, CD-ROMs and magnetic-optical disks, read-only memories (ROMs), random access memories (RAMs), EPROMS, EEPROMs, magnetic or optical cards, or any type of media suitable for storing electronic instructions.

The algorithms, methods, and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description above. In addition, the present embodiments are not described with reference to any particular programming language. It will be appreciated that a variety of programming languages may be used to implement the teachings of the present invention as described herein. It should also be noted that the terms “when” or the phrase “in response to,” as used herein, should be understood to indicate that there may be intervening time, intervening events, or both before the identified operation is performed.

Various operations are described as multiple discrete operations, in turn, in a manner that is most helpful in understanding the present disclosure, however, the order of description should not be construed to imply that these operations are necessarily order dependent. In particular, these operations need not be performed in the order of presentation.

Use of the phrase “configured to,” in one embodiment, refers to arranging, putting together, manufacturing, offering to sell, importing and/or designing an apparatus, hardware, logic, or element to perform a designated or determined task. In this example, an apparatus or element thereof that is not operating is still “configured to” perform a designated task if it is designed, coupled, and/or interconnected to perform said designated task.

Reference throughout this specification to “at least one embodiment” or “an embodiment” means that a particular feature, structure, or characteristic described in connection with the embodiment is included in at least one embodiment of the present disclosure. Thus, the appearances of the phrases “in at least one embodiment” or “in an embodiment” in various places throughout this specification are not necessarily all referring to the same embodiment. Furthermore, the particular features, structures, or characteristics can be combined in any suitable manner in one or more embodiments.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

What is claimed is:

1. A method of estimating peripheral oxygen saturation (SpO2) in a human subject, the method comprising:
capturing, by a camera, video comprising a plurality of image frames of the face and/or a hand palm of the human subject in the presence of an ambient light source;
identifying, within the plurality of image frames, a plurality of skin pixels corresponding pixels that spatially correspond to skin of the face and/or the hand palm within each of the plurality of image frames;
computing, for each of the plurality of skin pixels, a time-dependent signal corresponding to blood volume change for each color channel of the skin pixel;
generating, based on the time-dependent signal, a plurality of SpO2 estimates for each of the plurality of skin pixels at each frame of the captured video; and
computing an overall SpO2 estimate from the plurality of SpO2 estimates.

2. The method of claim 1, wherein generating the plurality of SpO2 estimates comprises, for each of the plurality of skin pixels, applying as inputs to a machine learning model a plurality of parameters derived from the corresponding time-dependent signal, and wherein the machine learning model comprises a multilayer perceptron model comprising at least three hidden layers.

3. The method of claim 1, wherein, prior to capturing the video, one or more performance settings of the camera are disabled, wherein the one or more performance settings are selected from bad pixel correction, Bayer domain hardware noise reduction, high-order lens-shading compensation, auto-white-balance, de-mosaic filtering, 3×3 color transformation, color artifact suppression, downscaling, or edge enhancement.

4. The method of claim 1, wherein identifying the plurality of skin pixels within the one or more image frames comprises detecting the face and/or the hand palm and extracting face landmarks and/or hand landmarks to determine bounds of the skin.

5. The method of claim 1, wherein computing, for each of the plurality of skin pixels, the time-dependent signal corresponding to blood volume change for each color channel of the skin pixel comprises computing the time-dependent signal for each of a red-channel, a green-channel, and a blue-channel of each skin pixel.

6. The method of claim 1, wherein computing the overall SpO2 estimate from the plurality of SpO2 estimates comprising applying a statistical inference to the plurality of SpO2 estimates computed over all of the identified skin pixels within each of the plurality of image frames of the captured video.

7. The method of claim 1, wherein, prior to capturing the video, increasing a color depth of video capture to 10 or more bits per color channel.

8. A device to estimate peripheral oxygen saturation (SpO2) in a human subject, the device comprising:
a housing;
a camera disposed at least partially in the housing;
a processing device disposed within the housing, the processing device being communicatively coupled to camera, wherein the processing device is configured to:
cause the camera to capture video comprising a plurality of image frames of the face and/or a hand palm of the human subject in the presence of an ambient light source;
identify, within the plurality of image frames, a plurality of skin pixels corresponding pixels that spatially correspond to skin of the face and/or the hand palm within each of the plurality of image frames;
compute, for each of the plurality of skin pixels, a time-dependent signal corresponding to blood volume change for each color channel of the skin pixel;
generate, based on the time-dependent signal, a plurality of SpO2 estimates for each of the plurality of skin pixels at each frame of the captured video; and
compute an overall SpO2 estimate from the plurality of SpO2 estimates; and
a display device configured to display the overall SpO2.

9. The device of claim 8, wherein generating the plurality of SpO2 estimates comprises, for each of the plurality of skin pixels, applying as inputs to a machine learning model a plurality of parameters derived from the corresponding time-dependent signal, wherein the machine learning model comprises a multilayer perceptron model comprising at least three hidden layers.

10. The device of claim 8, wherein, prior to capturing the video, one or more performance settings of the camera are disabled, wherein the one or more performance settings are selected from bad pixel correction, Bayer domain hardware noise reduction, high-order lens-shading compensation, auto-white-balance, de-mosaic filtering, 3×3 color transformation, color artifact suppression, downscaling, or edge enhancement.

11. The device of claim 8, wherein identifying the plurality of skin pixels within the one or more image frames comprises detecting the face and/or the hand palm and extracting face landmarks and/or hand landmarks to determine bounds of the skin.

12. The device of claim 8, wherein computing, for each of the plurality of skin pixels, the time-dependent signal corresponding to blood volume change for each color channel of the skin pixel comprises computing the time-dependent signal for each of a red-channel, a green-channel, and a blue-channel of each skin pixel.

13. The device of claim 8, wherein computing the overall SpO2 estimate from the plurality of SpO2 estimates comprising applying a statistical inference to the plurality of SpO2 estimates computed over all of the identified skin pixels within each of the plurality of image frames of the captured video.

14. The device of claim 8, wherein, prior to capturing the video, increasing a color depth of video capture to 10 or more bits per color channel.

15. A non-transitory computer-readable medium having instructions stored thereon, which, when executed by a processing device that is operatively coupled to a camera, causes the processing device to:
cause the camera to capture video comprising a plurality of image frames of the face and/or a hand palm of a human subject in the presence of an ambient light source;
identify, within the plurality of image frames, a plurality of skin pixels corresponding pixels that spatially correspond to skin of the face and/or the hand palm within each of the plurality of image frames;
compute, for each of the plurality of skin pixels, a time-dependent signal corresponding to blood volume change for each color channel of the skin pixel;
generate, based on the time-dependent signal, a plurality of SpO2 estimates for each of the plurality of skin pixels at each frame of the captured video; and
compute an overall SpO2 estimate from the plurality of SpO2 estimates.

16. The non-transitory computer-readable medium of claim 15, wherein generating the plurality of SpO2 estimates comprises, for each of the plurality of skin pixels, applying as inputs to a machine learning model a plurality of parameters derived from the corresponding time-dependent signal, wherein the machine learning model comprises a multilayer perceptron model comprising at least three hidden layers.

17. The non-transitory computer-readable medium of claim 15, wherein, prior to capturing the video, one or more performance settings of the camera are disabled, wherein the one or more performance settings are selected from bad pixel correction, Bayer domain hardware noise reduction, high-order lens-shading compensation, auto-white-balance, de-mosaic filtering, 3×3 color transformation, color artifact suppression, downscaling, or edge enhancement.

18. The non-transitory computer-readable medium of claim 15, wherein identifying the plurality of skin pixels within the one or more image frames comprises detecting the face and/or the hand palm and extracting face landmarks and/or hand landmarks to determine bounds of the skin.

19. The non-transitory computer-readable medium of claim 15, wherein computing, for each of the plurality of skin pixels, the time-dependent signal corresponding to blood volume change for each color channel of the skin pixel comprises computing the time-dependent signal for each of a red-channel, a green-channel, and a blue-channel of each skin pixel.

20. The non-transitory computer-readable medium of claim 15, wherein computing the overall SpO2 estimate from the plurality of SpO2 estimates comprising applying a statistical inference to the plurality of SpO2 estimates computed over all of the identified skin pixels within each of the plurality of image frames of the captured video, and wherein, prior to capturing the video, increasing a color depth of video capture to 10 or more bits per color channel.
